(19) United States
(12) Patent Application Publication
(10) Pub. No.: US 2023/0397907 A1
(43) Pub. Date: Dec. 14, 2023

(54) SUTURE RETENTION DEVICES

(71) Applicant: Timothy William Ingraham Clark, Philadelphia, PA (US)

(72) Inventor: Timothy William Ingraham Clark, Philadelphia, PA (US)

(21) Appl. No.: 18/209,082

(22) Filed: Jun. 13, 2023

Related U.S. Application Data
(60) Provisional application No. 63/351,844, filed on Jun. 14, 2022.

(51) Int. Cl. A61B 17/04

(52) U.S. Cl. CPC A61B 17/0401; A61B 2017/0424

(57) ABSTRACT

Suture retention devices are disclosed that are configured to grip one or more end portions of a suture at multiple locations. According to one aspect, at least first and second gripping locations are provided between a depressible first actuator and a wall of housing in which the first actuator resides. In addition, at least third and fourth gripping locations are provided between the depressible first actuator and a depressible second actuator located inside a cavity of the first actuator. According to another aspect, at least first and second gripping locations are provided as a result of an actuator rotating between first and second angular positions inside a housing of the suture retention device. According to another aspect, suture retention devices that include indica of the size of a puncture wound site are provided.

### CROSS-REFERENCE TO RELATED APPLICATIONS

This application relates to and claims the benefit and priority to U.S. provisional patent application 63/351,844, filed Jun. 14, 2022, which is incorporated herein by reference in its entirety.

### TECHNICAL FIELD

The present invention relates to devices used in the closure of percutaneous catheter and sheath insertion sites to achieve hemostasis.

### BACKGROUND

The use of sutures to close percutaneous insertion sites is known. Purse string sutures are commonly used for this purpose. A purse string suture typically includes a filament/thread that is stitched in a circular pattern around the insertion site. When the stitching is complete, first and second opposing ends of the suture extend from the skin of the patient adjacent one another. The manner in which the suture is stitched about the insertion site permits a practitioner to close the insertion site by merely pulling on the opposing end portions of the suture. Tension on the opposing end portions of the suture must be maintained for a period of time in order to achieve hemostasis. For this reason, devices have been developed to apply the requisite tension so that the practitioner is freed to perform other tasks.

### SUMMARY

Disclosed are suture retention devices used in the course of achieving hemostasis at a percutaneous catheter or sheath insertion site.

According to one implementation a suture retention device for releasably securing one or more end portions of a suture is provided, wherein the suture retention device comprises a housing having a distal end portion that includes a first inlet passage and a proximal end portion that includes a first outlet passage that is longitudinal aligned with the first inlet passage, the housing defining a first cavity disposed between and communicated with each of the first inlet and first outlet passages. The suture retention device also comprises first and second actuators. The first actuator is disposed inside the first cavity and moveable in the first cavity between a first fully depressed position and a first undepressed position, the first actuator being continuously biased towards the first undepressed position and including a distal end portion that includes a second inlet passage and a proximal end portion that includes a second outlet passage that is longitudinally aligned with the second inlet passage, the first actuator defining a second cavity disposed between and communicated with each of the second inlet and second outlet passages, when the first actuator is in the first undepressed position the second inlet and second outlet passages are respectively not longitudinally aligned with the first inlet and first outlet passages of the housing, when the first actuator is in the first fully depressed position the second inlet and second outlet passages are respectively longitudinally aligned with the first inlet and first outlet passages of the housing. The second actuator is disposed inside the second cavity and moveable in the second cavity between a second fully depressed position and a second undepressed position, the second actuator being continuously biased towards the second undepressed position and including a longitudinal through passage that extends between a distal end and a proximal end of the second actuator, when the second actuator is in the second undepressed position the longitudinal through passage is not longitudinally aligned with either of the second inlet and second outlet passages of the first actuator, when the second actuator is in the second fully depressed position the longitudinal through passage is longitudinally aligned with the second inlet and second outlet passages of the first actuator.

According to some implementations the first actuator is continuously biased towards the first undepressed position by a first biasing member and the second actuator is continuously biased towards the second undepressed position by a second biasing member. According to some implementations the second biasing member is surrounded by the first biasing member. According to some implementations each of the first and second biasing members is a coil spring.

According to some implementations the housing and the first actuator are configured such that when the first actuator is in the first undepressed position, the one or more end portions of the suture, when situated in the suture retention device, is/are gripped between the housing and the first actuator at first and second locations. According to some implementations a portion of the housing and/or a portion of the first actuator comprises a roughened surface in a region of one or both the first and second locations.

According to some implementations the first and second actuators are configured such that when the second actuator is in the first undepressed position, the one or more end portions of the suture, when situated in the suture retention device, is/are gripped between the first and second actuator at third and fourth locations. According to some implementations the first and second locations are spaced-apart and the third and fourth locations are spaced apart, and each of the third and fourth locations are different than the first and second locations. According to some implementations a portion of the first actuator and/or a portion of the second actuator comprises a roughened surface in a region of one or both the third and fourth locations.

According to some implementations the suture retention device also includes a threading assembly that includes a looped filament extending through the suture retention device. The looped filament has a proximal end located proximal the first outlet passage of the housing and a distal end located distal to the first distal passage of the housing.

According to some implementations, when the first actuator is in the first undepressed state and the second actuator is in the second undepressed state, a top of the second actuator is located above a top of the first actuator.

According to some implementations the first actuator is configured to rotate inside the first cavity between a first angular position when in the first fully depressed state and a second angular position different from the first angular position when in the first undepressed state. According to some implementations the second actuator is coupled to the first actuator and rotates with the first actuator. According to some implementations the housing and the first actuator are configured such that when the first actuator is in the second angular position, the one or more end portions of the suture, when situated in the suture retention device, is/are gripped between the housing the first actuator.

According to some implementations the first actuator includes one or more protrusions protruding from a side surface, and an internal wall of the housing includes a respective one or more angular grooves in which the one or more protrusions reside, the one or more protrusions and the one or more angular grooves being configured to guide the first actuator between the first and second angular positions. In alternative implementations, the internal wall of the housing includes one or more protrusions and the first actuator includes a side surface having one or more angular grooves in which the one or more protrusions reside, the one or more protrusions and the one or more angular grooves being configured to guide the first actuator between the first and second angular positions. According to some implementations the one or more protrusions are snap-fitted into the respective one or more grooves.

According to some implementations the second actuator is configured to rotate inside the second cavity between a first angular position when in the second fully depressed state and a second angular position different from the first angular position when in the second undepressed state. According to some implementations the first and second actuators are configured such that when the second actuator is in the second angular position, the one or more end portions of the suture, when situated in the suture retention device, is/are gripped between the first and second actuators.

According to another implementation a suture retention device for releasably securing one or more end portions of a suture is provided that includes a housing and an actuator disposed inside a cavity of the housing. The housing has a distal end portion that includes an inlet passage and a proximal end portion that includes an outlet passage that is longitudinal aligned with the inlet passage. The cavity of the housing is disposed between and communicated with each of the inlet and outlet passages. The actuator includes a through passage and is configured to rotate inside the cavity between a first angular position and a second angular position. When in the first angular position, the through passage is longitudinal aligned with each of the inlet and outlet passages of the housing, and when in the second angular position, the through passage is not longitudinally aligned with the inlet and outlet passages of the housing. When the actuator is in the second angular position, the one or more end portions of the suture, when situated in the suture retention device, is/are gripped between the housing and the actuator at first and second spaced-apart locations. According to some implementations, a portion of the housing and/or the actuator comprises a roughened surface in a region of one or both of the first and second spaced-apart locations.

According to some implementations the actuator is continuously biased towards the second angular position. According to some implementations the actuator is continuously biased towards the second angular position by a spring coil positioned between a bottom surface of the cavity of the housing and a bottom of the actuator.

According to some implementations the actuator includes one or more protrusions protruding from a side surface thereof, and an internal wall of the housing includes a respective one or more angular grooves in which the one or more protrusions reside. The one or more protrusions and the one or more angular grooves are configured to guide the actuator between the first and second angular positions. According to some implementations the one or more protrusions are snap-fitted into the respective one or more grooves.

According to other implementations an internal wall of the housing includes one or more protrusions and the actuator includes a side surface having one or more angular grooves in which the one or more protrusions reside. The one or more protrusions and the one or more angular grooves are configured to guide the actuator between the first and second angular positions. According to some implementations the one or more protrusions are snap-fitted into the respective one or more grooves.

According to some implementations the actuator is moveable in the cavity of the housing between a fully depressed position and aN undepressed position. The actuator is continuously biased towards the undepressed position and includes a longitudinal through passage that extends between a distal end portion and a proximal end portion of the actuator. When the actuator is in the undepressed position the longitudinal through passage is not longitudinally aligned with the inlet and outlet passages of the housing, and when the actuator is in the fully depressed position the longitudinal through passage is longitudinally aligned with the inlet and outlet passages of the housing. The housing and the actuator are configured such that when the actuator is in the first undepressed state, the one or more end portions of the suture, when situated in the suture retention device, is/are gripped between the housing and the actuator at two locations. According to some implementations a portion of the housing and/or the actuator comprises a roughened surface in a region of one or both of the two locations.

These and other advantages and features will become apparent in view of the figures and the detailed description.

### BRIEF DESCRIPTION OF THE DRAWINGS

FIG. 1 is a perspective view of a suture retention device according to one implementation.

FIG. 2 is an exploded view of the suture retention device of FIG. 1.

FIG. 3A is a cross-section view of the suture retention device of FIG. 1 along line A-A with each of the first and second actuators in an undepressed position.

FIG. 3B is a cross-section view of the suture retention device of FIG. 1 along line A-A with each of the first and second actuators in a fully depressed position.

FIG. 4 is a cross-section side view of the suture retention device of FIG. 1 with each of the first and second actuators in an undepressed position.

FIG. 5A is a cross-section side view of the suture retention device of FIG. 1 with each of the first and second actuators in a fully depressed position.

FIG. 5B is a perspective view of the suture retention device shown in FIG. 5A

FIG. 6A shows the suture retention device of FIG. 5A with opposing end portions of a suture extending through the device.

FIG. 6B shows the suture retention device of FIG. 5B with opposing end portions of a suture extending through the device.

FIG. 7 shows the suture retention device of FIG. 6A after the first and second actuators resume an undepressed state.

FIG. 8 is a perspective view of a suture retention device according to another implementation.

FIG. 9 is a first cut-away view of the housing shown in FIG. 8.

FIG. 10 is a second cut-away view of the housing shown in FIG. 8

FIG. 11A is a cross-section top view of the housing and actuator of the suture retention device of FIG. 8.

FIG. 11B is a cross-section top view of the housing and actuator of the suture retention device of FIG. 8, showing a suture residing therein.

FIG. 12A is a cross-section side view of the housing and actuator of the suture retention device of FIG. 8.

FIG. 12B is a cross-section side view of the housing and actuator of the suture retention device of FIG. 8, showing a suture residing therein.

FIG. 13 illustrates a positioning of a suture in the suture retention device of FIG. 1 after a rotating of the actuator in the housing.

FIG. 14 illustrates a tortuous path of a tensioned suture inside the suture retention device of FIG. 1

### DETAILED DESCRIPTION

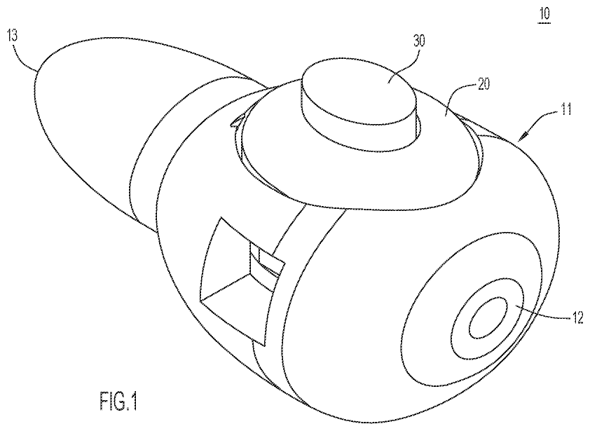

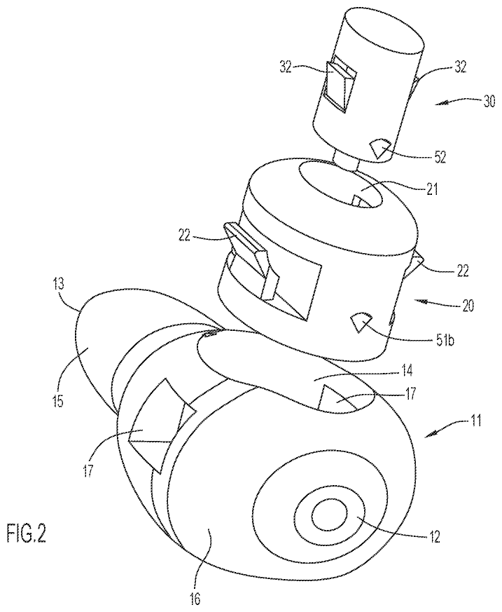

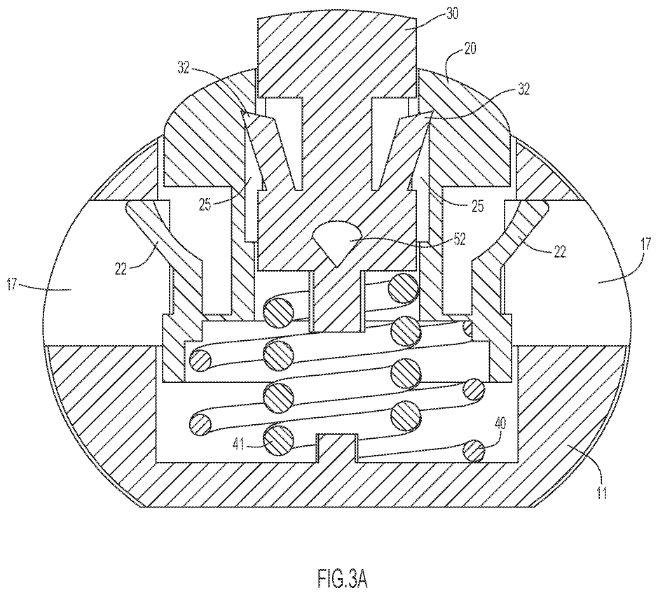

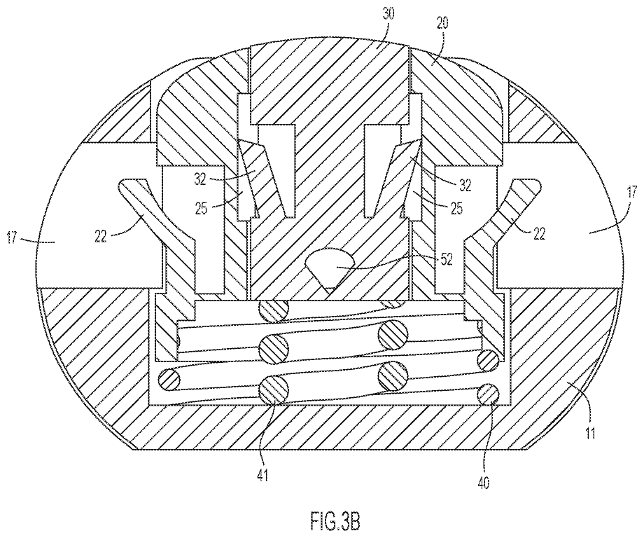

FIG. 1 is a perspective view of a suture retention device 10 according to one implementation. The device includes a housing 11 having a proximal end 12 and a distal end 13. The device also includes a first actuator 20 and a second actuator 30 that are each configured to assume an undepressed position (as shown in FIGS. 1, 3A, 4 and 7) and also configured to assume a fully depressed position (as shown in FIGS. 3B, 5A, 5B, 6A and 6B). FIG. 2 is an exploded view of the suture retention device of FIG. 1 in which each of the housing 11, first actuator 20 and second actuator 30 are identified. In an assembled state, at least a bottom portion of the first actuator 20 is disposed inside a cavity 14 of the housing 11, and at least a bottom portion of the second actuator 30 is disposed inside a cavity 21 of the first actuator 20. As shown in FIGS. 1, 3A, 4 and 7, when the first actuator 20 is in the first undepressed state and the second actuator 20 is in the second undepressed state, a top of the second actuator is located above a top of the first actuator.

The housing has an elongate distal end portion 15 that narrows towards the distal end so that a line of site may be maintained with the percutaneous insertion site while the suture retention device is in use. The housing 11 includes an inlet passage 50a that extends through the elongate distal end portion 15. The housing 11 also includes a first outlet passage 50b that extends through the proximal end portion 16 and is aligned along a longitudinal axis L with inlet passage 50a. The housing cavity 14 is disposed between and communicated with each of the inlet and outlet passages 50a and 50b.

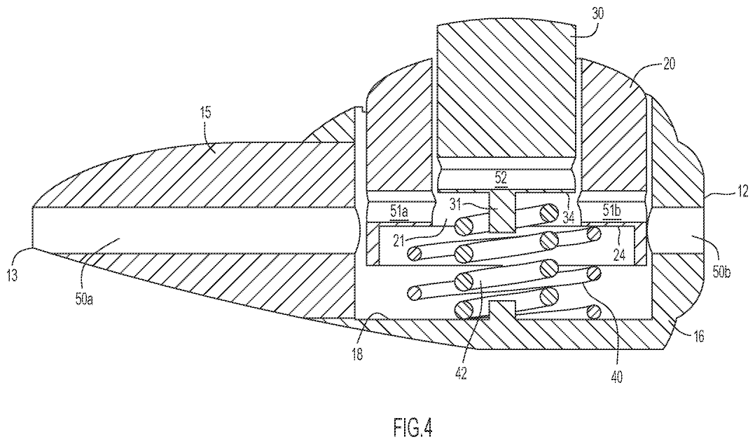

As noted above, the first actuator 20 is disposed inside the housing cavity 14 cavity and is moveable in the cavity between a fully depressed position (as shown in FIGS. 3B, 5A, 5B, 6A and 6B) and an undepressed position (as shown in FIGS. 1, 3A, 4 and 7). The device 10 also includes a first biasing member 40 (e.g. coil spring) that is position between a bottom surface 18 of the housing 11 and a bottom segment 24 of the first actuator 20. The first biasing member functions to continuously urge the first actuator towards the undepressed position. As shown in FIGS. 4-7, the first actuator 20 includes a distal end portion that includes an inlet passage 51a and a proximal end portion that includes an outlet passage 51b that is longitudinally aligned with inlet passage 51a. The cavity 21 of the first actuator 20 is disposed between and communicated with each of the inlet and outlet passages 51a and 51b.

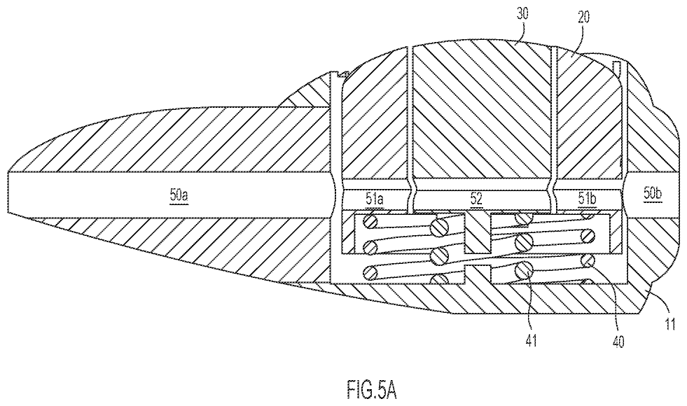

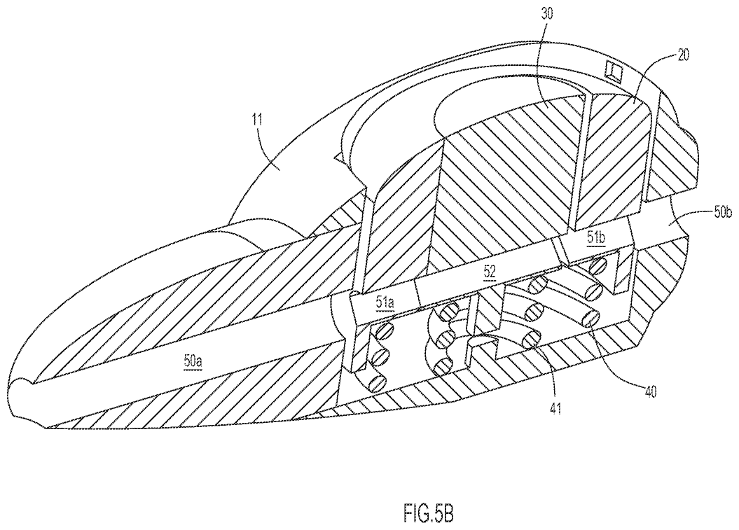

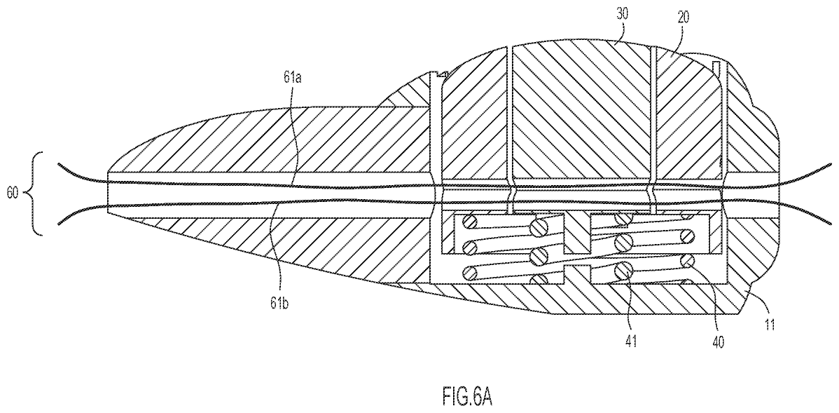

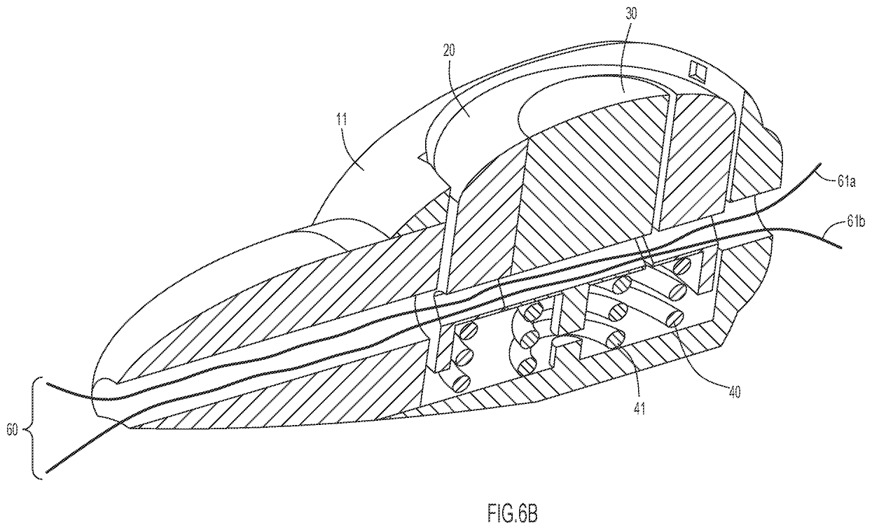

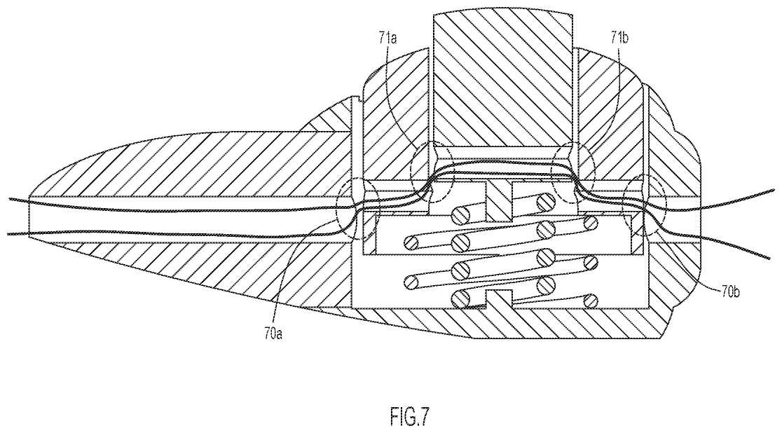

When the first actuator 20 is in the undepressed position, inlet passage 51a and outlet passage 51b are respectively not longitudinally aligned with inlet passage 50a and outlet passage 50b of the housing 11 as shown in FIGS. 4 and 7. However, when the first actuator 20 is in the fully depressed position the inlet and outlet passages 51a, 51b are respectively longitudinally aligned with the inlet and outlet passages 50a, 50b of the housing 11 as shown in FIGS. 5A-6B.

As explained above, the second actuator 30 is disposed inside the cavity 21 of the first actuator 20 and is moveable in cavity 21 between a fully depressed position and an undepressed position. The device 10 also includes a second biasing member 41 (e.g. coil spring) that is position between the bottom surface 18 of the housing 11 and a bottom segment 34 of the second actuator 20. The second biasing member 41 functions to continuously urge the second actuator 30 towards the undepressed position. According to some implementations the second biasing member 41 is surrounded by the first biasing member 40.

The second actuator 30 includes a longitudinal through passage 52 that extends between proximal and distal ends of the second actuator. When the second actuator is in the undepressed position the longitudinal through passage 52 is not longitudinally aligned with either of the inlet and outlet passages 51a, 51b of the first actuator 20 as shown in FIGS. 4 and 7. However, when the second actuator 30 is in the fully depressed position the longitudinal through passage 52 is longitudinally aligned with the inlet and outlet passages 51a and 51b of the first actuator 20.

As a result of the above-disclosed configuration, when each of the first and second actuators is in its fully depressed position, all of passages 50a, 50b, 51a, 51b and 52 are longitudinally aligned as shown in FIGS. 6A and 6B such that end portions 61a and 61b of a suture 60 are capable of being advanced through a full length of the device 10 with little resistance. The advancement of the suture 60 through the retention device 10 is typically achieved by use of a threading assembly that is preloaded in the device 10. According to one implementation the threading assembly includes a looped filament that originally extends through the suture retention device 10. The looped filament typically includes a proximal end located proximal the outlet passage 51b of the housing 11 and a distal end located distal to the distal passage 51a of the housing 11.

Upon the end portions 61a and 61b of suture 60 being fully advanced through the device 10 with tension applied thereto to cause a closure of a percutaneous puncture/insertion site, the tension may be sustained by a releasing of the first and second actuators to allow them to assume their undepressed positions. As illustrated in FIG. 7, the releasing of the first actuator 20 causes a longitudinal misalignment among housing passages 50a, 50b and passages 51a and 51b of the first actuator 20. This results in the suture 60 being gripped between the housing 11 and the first actuator 20 at least two spaced-apart locations 70a and 70b by an exertion of force applied by the first biasing member 40. With continued reference to FIG. 7, the releasing of the second actuator causes a longitudinal misalignment among passage 52 and passages 51a and 51b of the first actuator 20. This results in the suture 60 being gripped between the first actuator 20 and the second actuator 30 at at least two spaced-apart locations 71a and 71b by an exertion of force applied by the second biasing member 41. The creation of the at least four spaced-apart gripping locations 70a, 70b, 71a and 71b on the suture 60 advantageously allows a larger overall force to be applied to the suture 60 to maintain its tension without the need to employ stiffer and more forceful springs. Good suture tensioning can thus be maintained without the risk of the suture being severed by excessive force.

According to some implementations a portion of the housing 11 and/or a portion of the first actuator 20 comprises a roughened surface in a region of one or both of gripping locations and 70b. The roughened surfaces further inhibit a sliding of the suture 60 at the gripping locations 70a and 70b.

According to some implementations, a portion of the first actuator and/or a portion of the second actuator comprises a roughened surface in a region of one or both of the gripping locations 71a and 71b. The roughened surfaces further inhibit a sliding of the suture 60 at the gripping locations 71a and 71b.

As shown in FIGS. 2, 3A and 3B, the housing 11 and first actuator 20 comprise features that couple the first actuator to the housing in a sliding manner. According to some implementations these features include hinged and protruding tabs 22 on opposite sides of the first actuator 20 that fit inside and are slidable in grooves or windows 17 of the housing 11. As also shown in FIGS. 2, 3A and 3B, the first actuator 20 and second actuator 30 comprise features that couple the second actuator to the first actuator in a sliding manner. According to some implementations these features include hinged and protruding tabs 32 on opposite sides of the second actuator 30 that fit inside and are slidable in grooves or windows 25 of the first actuator 20.

According to some implementations the first actuator 20 has a circular profile and is configured to rotate inside the housing cavity 14 between a first angular position when in the fully depressed position and a second angular position different from the first angular position when in the undepressed position. According to some implementations the second actuator 30 rotates with the first actuator 20. As will be discussed in more detail below, an angular rotation of an actuator inside the housing can provide additional gripping of the suture 60, and in the present case between the housing 11 and the first actuator 20. In conjunction with the foregoing, or alternatively, the second actuator 30 may be configured to rotate inside the cavity 21 of the first actuator 20 between a first angular position when the second actuator is in the fully depressed position and a second angular position different from the first angular position when in the undepressed position.

In implementations wherein the first actuator 20 rotates with respect to the housing when transitioning between the undepressed position and fully depressed position, the first actuator may comprise one or more protrusions protruding from a side surface thereof, and an internal wall of the housing may comprise a respective one or more angular grooves in which the one or more protrusions reside. In such a case, the one or more protrusions and the one or more angular grooves are configured to guide the first actuator 20 between the first and second angular positions. According to some implementations the one or more protrusions are snap-fitted into the respective one or more grooves.

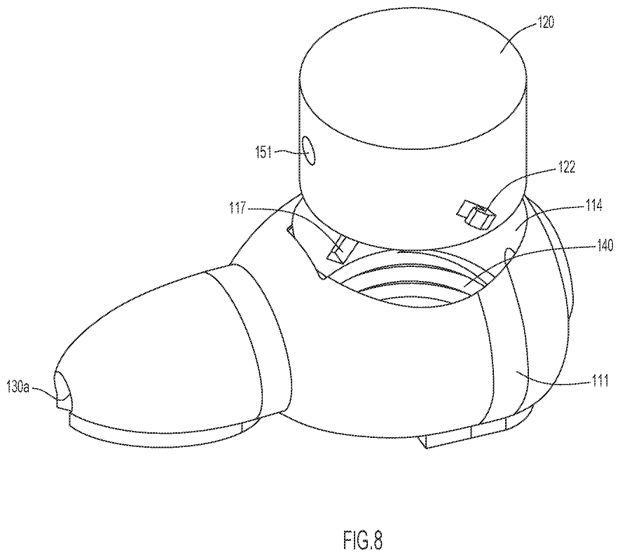

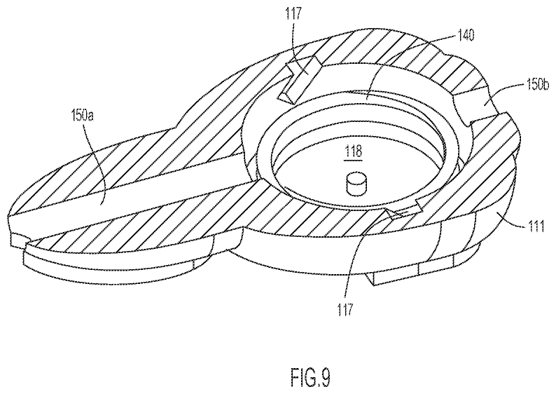

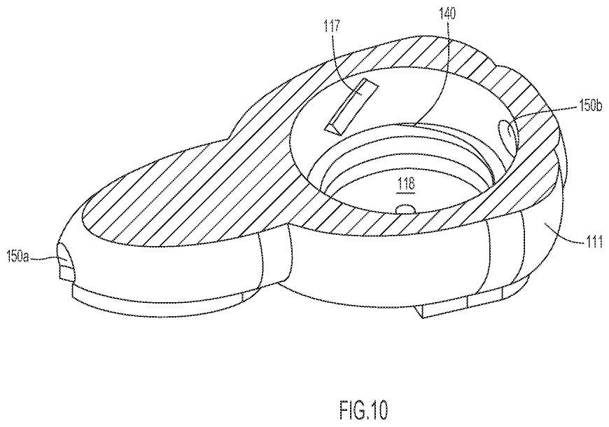

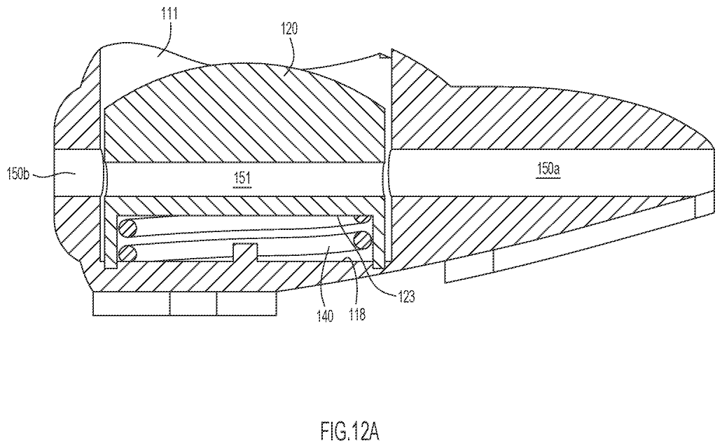

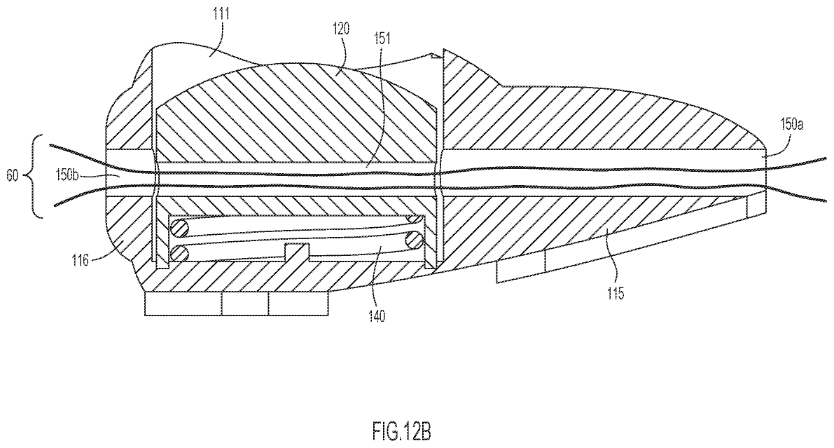

FIGS. 8 through 14 illustrate implementations of another suture retention device 100 that is configured to releasably securing one or more end portions of a suture 60 in a tensioned state. Device 100 includes a housing 111 having a distal end portion 115 that includes an inlet passage 150a and a proximal end portion 116 that includes an outlet passage 150b that is longitudinal aligned with inlet passage 150a. The housing 111 defines a cavity 114 disposed between and communicated with each of the inlet and outlet passages 150a and 150b.

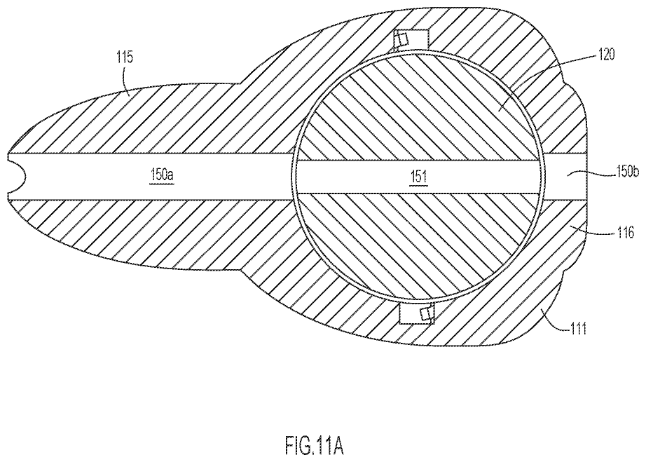

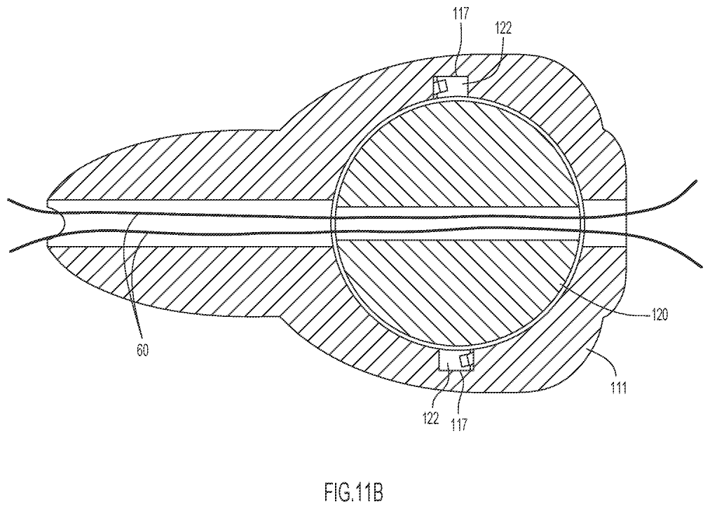

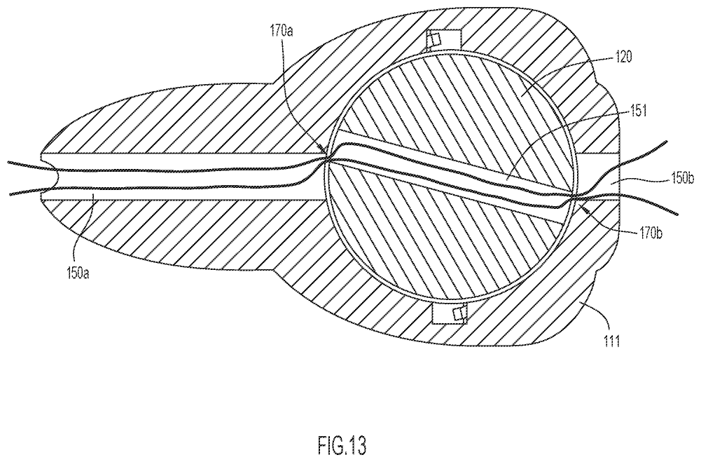

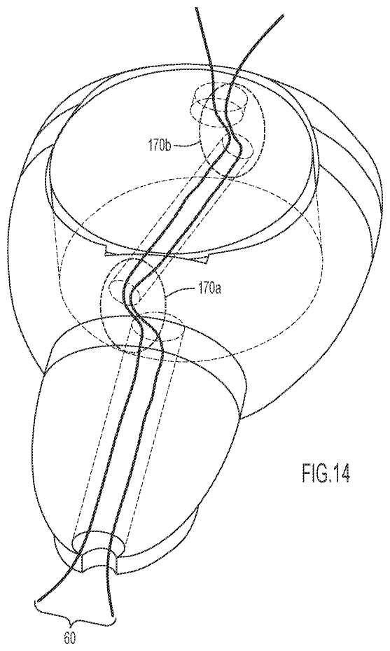

Device 100 also includes an actuator 120 disposed inside the cavity 114 of the housing 111. The actuator 120 includes a through passage 151 and is configured to rotate inside the cavity 114 between a first angular position (as shown in FIGS. 11A and 11B) and a second angular position (as shown in FIGS. 13 and 14). When the actuator 120 is in the first angular position the through passage 151 is longitudinal aligned with each of the inlet and outlet passages 150a and 150b of the housing 111 to allow a relatively free advancement of the suture 60 through an entire length of the device 100. However, when the actuator 120 is in the second angular position the through passage 151 is not longitudinally aligned with the inlet and outlet passages 150a and 150b of the housing 111, which results in the suture 60 being gripped between the housing 111 and the actuator 120 at first and second spaced-apart locations 170a and 170b as shown in FIGS. 13 and 14. According to some implementations the actuator 120 is continuously biased towards the second angular position by a biasing member 140 (e.g. coil spring) positioned between a bottom surface 118 of cavity 114 and a bottom region 123 of the actuator 120.

According to some implementations, the actuator includes one or more (and preferably two or more) protrusions 122 that each protrude from side surfaces thereof, and an internal wall of the housing 111 includes a respective one or more angular grooves 117 in which the one or more protrusions reside. The protrusions 122 and the angular grooves 117 are configured to guide the actuator 120 between the first and second angular positions. According to other implementations the internal wall of the housing 111 includes one or more protrusions and the actuator 120 includes a side surface having one or more angular grooves in which the one or more protrusions reside. The one or more protrusions and the one or more angular grooves being configured to guide the actuator 120 between the first and second angular positions. In either case, according to some implementations the one or more protrusions are hinged elements that are snap-fitted into the respective one or more grooves.

In a manner consistent with the implementations disclosed above in conjunction with FIGS. 1-8, according to some implementations the actuator 120 of suture retention device 100 is additionally arranged moveable in the cavity 114 of the housing 111 between a fully depressed position and an undepressed position. According to some implementations the actuator 120 is continuously biased towards the undepressed position and when the actuator is in the undepressed position the longitudinal through passage 151 is located above/vertically offset from the inlet and outlet passages 150a and 150b of the housing 111. However, when the actuator 120 is in the fully depressed position, the longitudinal through passage 151 is longitudinally and vertically aligned with the inlet and outlet passages 150a, 150b of the housing 111.

According to some implementations the housing 111 and the actuator 120 are configured such that when the actuator 120 is in the undepressed position, one or more end portions of the suture 60, when situated in the suture retention device 100, is/are gripped between the housing 111 and the actuator 120 at at least two locations.

Similar or additional implementations are disclosed in the clauses that follow.
Clause 1. A suture retention device for releasably securing one or more end portions of a suture, the suture retention device comprising;
   a housing having a distal end portion that includes a first inlet passage and a proximal end portion that includes a first outlet passage that is longitudinal aligned with the first inlet passage, the housing defining a first cavity disposed between and communicated with each of the first inlet and first outlet passages;
   a first actuator disposed inside the first cavity and moveable in the first cavity between a first fully depressed position and a first undepressed position, the first actuator being continuously biased towards the first undepressed position and including a distal end portion that includes a second inlet passage and a proximal end portion that includes a second outlet passage that is longitudinally aligned with the second inlet passage, the first actuator defining a second cavity disposed between and communicated with each of the second inlet and second outlet passages, when the first actuator is in the first undepressed position the second inlet and second outlet passages are respectively not longitudinally aligned with the first inlet and first outlet passages of the housing, when the first actuator is in the first fully depressed position the second inlet and second outlet passages are respectively longitudinally aligned with the first inlet and first outlet passages of the housing; and
   a second actuator disposed inside the second cavity and moveable in the second cavity between a second fully depressed position and a second undepressed position, the second actuator being continuously biased towards the second undepressed position and including a longitudinal through passage that extends between a distal end and a proximal end of the second actuator, when the second actuator is in the second undepressed position the longitudinal through passage is not longitudinally aligned with either of the second inlet and second outlet passages of the first actuator, when the second actuator is in the second fully depressed position the longitudinal through passage is longitudinally aligned with the second inlet and second outlet passages of the first actuator.
Clause 2. The suture retention device according to clause 1, wherein the first actuator is continuously biased towards the first undepressed state by a first biasing member and the second actuator is continuously biased towards the second undepressed state by a second biasing member.
Clause 3. The suture retention device according to clause 2, wherein the housing and the first actuator are configured such that when the first actuator is in the first undepressed state, the one or more end portions of the suture, when situated in the suture retention device, is/are gripped between the housing and the first actuator at first and second locations.
Clause 4. The suture retention device according to clause 3, wherein the first and second actuators are configured such that when the second actuator is in the first undepressed state, the one or more end portions of the suture, when situated in the suture retention device, is/are gripped between the first and second actuator at third and fourth locations.
Clause 5. The suture retention device according to clause 4, wherein the first and second locations are spaced-apart and the third and fourth locations are spaced apart, each of the third and fourth locations being different than the first and second locations.
Clause 6. The suture retention device according to clause 2, wherein the second biasing member is surrounded by the first biasing member.
Clause 7. The suture retention device according to clause 6, wherein each of the first and second biasing members is a coil spring.
Clause 8. The suture retention device according to clause 1, further comprising a threading assembly that includes a looped filament extending through the suture retention device and having a proximal end located proximal the first outlet passage of the housing and a distal end located distal to the first distal passage of the housing.
Clause 9. The suture retention device according to clause 1, wherein when the first actuator is in the first undepressed position and the second actuator is in the second undepressed position, a top of the second actuator is located above a top of the first actuator.
Clause 10. The suture retention device according to clause 1, wherein the first actuator is configured to rotate inside the first cavity between a first angular position when in the first fully depressed position and a second angular position different from the first angular position when in the first undepressed position.
Clause 11. The suture retention device according to clause 1, wherein the second actuator is coupled to the first actuator and rotates with the first actuator.
Clause 12. The suture retention device according to clause 1, wherein the second actuator is configured to rotate inside the second cavity between a first angular position when in the second fully depressed position and a second angular position different from the first angular position when in the second undepressed position.
Clause 13. The suture retention device according to clause 10, wherein the housing and the first actuator are configured such that when the first actuator is in the second angular position, the one or more end portions of the suture, when situated in the suture retention device, is/are gripped between the housing and the first actuator.
Clause 14. The suture retention device according to clause 12, wherein the first and second actuators are configured such that when the second actuator is in the second angular position, the one or more end portions of the suture, when situated in the suture retention device, is/are gripped between the first and second actuators.
Clause 15. The suture retention device according to clause 10, wherein the first actuator includes one or more protrusions protruding from a side surface, and an internal wall of the housing includes a respective one or more angular grooves in which the one or more protrusions reside, the one or more protrusions and the one or more angular grooves being configured to guide the first actuator between the first and second angular positions.
Clause 16. The suture retention device according to clause 10, wherein an internal wall of the housing includes one or more protrusions, the first actuator includes a side surface having one or more angular grooves in which the one or more protrusions reside, the one or more protrusions and the one or more angular grooves being configured to guide the first actuator between the first and second angular positions.
Clause 17. The suture retention device according to clause 15, wherein the one or more protrusions are snap-fitted into the respective one or more grooves.
Clause 18. The suture retention device according to clause 16, wherein the one or more protrusions are snap-fitted into the respective one or more grooves.
Clause 19. The suture retention device according to clause 3, wherein a portion of the housing and/or a portion of the first actuator comprises a roughened surface in a region of one or both of the first and second locations.
Clause 20. The suture retention device according to clause 4, wherein a portion of the first actuator and/or a portion of the second actuator comprises a roughened surface in a region of one or both of the third and fourth locations.

Additional limitations are disclosed in the clauses that follow.
Clause 1. A suture retention device for releasably securing one or more end portions of a suture, the suture retention device comprising;
   a housing having a distal end portion that includes an inlet passage and a proximal end portion that includes an outlet passage that is longitudinal aligned with the inlet passage, the housing defining a cavity disposed between and communicated with each of the inlet and outlet passages; and
   an actuator disposed inside the cavity of the housing, the actuator including a through passage and configured to rotate inside the cavity between a first angular position and a second angular position, when in the first angular position the through passage is longitudinal aligned with each of the inlet and outlet passages of the housing, when in the second angular position the through passage is not longitudinally aligned with the inlet and outlet passages of the housing, when the actuator is in the second angular position, the one or more end portions of the suture, when situated in the suture retention device, is/are gripped between the housing and the actuator at first and second spaced-apart locations.
Clause 2. The suture retention device according to clause 1, wherein the actuator is continuously biased towards the second angular position.
Clause 3. The suture retention device according to clause 1, wherein the actuator includes one or more protrusions protruding from a side surface thereof, and an internal wall of the housing includes a respective one or more angular grooves in which the one or more protrusions reside, the one or more protrusions and the one or more angular grooves being configured to guide the actuator between the first and second angular positions.
Clause 4. The suture retention device according to clause 1, wherein an internal wall of the housing includes one or more protrusions, the actuator including a side surface having one or more angular grooves in which the one or more protrusions reside, the one or more protrusions and the one or more angular grooves being configured to guide the actuator between the first and second angular positions.
Clause 5. The suture retention device according to clause 3, wherein the one or more protrusions are snap-fitted into the respective one or more grooves.
Clause 6. The suture retention device according to clause 4, wherein the one or more protrusions are snap-fitted into the respective one or more grooves.
Clause 7. The suture retention device according to clause 1, wherein the actuator is moveable in the cavity of the housing between a fully depressed position and a undepressed position, the actuator being continuously biased towards the undepressed position and including a longitudinal through passage that extends between a distal end portion and a proximal end portion of the actuator, when the actuator is in the undepressed position the longitudinal through passage is not longitudinally aligned with the inlet and outlet passages of the housing, when the actuator is in the fully depressed position the longitudinal through passage is longitudinally aligned with the inlet and outlet passages of the housing.
Clause 8. The suture retention device according to clause 7, wherein the housing and the actuator are configured such that when the actuator is in the undepressed position, the one or more end portions of the suture, when situated in the suture retention device, is/are gripped between the housing and the actuator at two locations.
Clause 9. The suture retention device according to clause 1, wherein a portion of the housing and/or the actuator comprises a roughened surface in a region of one or both of the first and second spaced-apart locations.
Clause 10. The suture retention device according to clause 8, wherein a portion of the housing and/or the actuator comprises a roughened surface in a region of one or both of the two locations.
Clause 11. The suture retention device according to clause 2, wherein the actuator is continuously biased towards the second angular position by a spring coil positioned between a bottom surface of the cavity of the housing and a bottom of the actuator.

Additional limitations are disclosed in the clauses that follow.
Clause 1. A suture retention device for releasably securing one or more end portions of a suture used to close a percutaneous insertion site of a catheter or sheath, the suture retention device including indica of the size of the percutaneous insertion site.
Clause 2. The suture retention device according to clause 1, wherein the indica is a marking indicative of a size or range of sizes of the catheter or the sheath.
Clause 3. The suture retention device according to clause 1, wherein the indica is a coloring of at least a part of the suture retention device.
Clause 4. The suture retention device according to clause 2, wherein the marking is a French size or range of French sizes of the catheter or sheath.

What is claimed is:

1. A suture retention device for releasably securing one or more end portions of a suture, the suture retention device comprising;
a housing having a distal end portion that includes a first inlet passage and a proximal end portion that includes a first outlet passage that is longitudinal aligned with the first inlet passage, the housing defining a first cavity disposed between and communicated with each of the first inlet and first outlet passages;
a first actuator disposed inside the first cavity and moveable in the first cavity between a first fully depressed position and a first undepressed position, the first actuator being continuously biased towards the first undepressed position and including a distal end portion that includes a second inlet passage and a proximal end portion that includes a second outlet passage that is longitudinally aligned with the second inlet passage, the first actuator defining a second cavity disposed between and communicated with each of the second inlet and second outlet passages, when the first actuator is in the first undepressed position the second inlet and second outlet passages are respectively not longitudinally aligned with the first inlet and first outlet passages of the housing, when the first actuator is in the first fully depressed position the second inlet and second outlet passages are respectively longitudinally aligned with the first inlet and first outlet passages of the housing; and
a second actuator disposed inside the second cavity and moveable in the second cavity between a second fully depressed position and a second undepressed position, the second actuator being continuously biased towards the second undepressed position and including a longitudinal through passage that extends between a distal end and a proximal end of the second actuator, when the second actuator is in the second undepressed position the longitudinal through passage is not longitudinally aligned with either of the second inlet and second outlet passages of the first actuator, when the second actuator is in the second fully depressed position the longitudinal through passage is longitudinally aligned with the second inlet and second outlet passages of the first actuator.

2. The suture retention device according to claim 1, wherein the first actuator is continuously biased towards the first undepressed state by a first biasing member and the second actuator is continuously biased towards the second undepressed state by a second biasing member.

3. The suture retention device according to claim 2, wherein the housing and the first actuator are configured such that when the first actuator is in the first undepressed state, the one or more end portions of the suture, when situated in the suture retention device, is/are gripped between the housing and the first actuator at first and second locations.

4. The suture retention device according to claim 3, wherein the first and second actuators are configured such that when the second actuator is in the first undepressed state, the one or more end portions of the suture, when situated in the suture retention device, is/are gripped between the first and second actuator at third and fourth locations.

5. The suture retention device according to claim 4, wherein the first and second locations are spaced-apart and the third and fourth locations are spaced apart, each of the third and fourth locations being different than the first and second locations.

6. The suture retention device according to claim 2, wherein the second biasing member is surrounded by the first biasing member.

7. The suture retention device according to claim 6, wherein each of the first and second biasing members is a coil spring.

8. The suture retention device according to claim 1, further comprising a threading assembly that includes a looped filament extending through the suture retention device and having a proximal end located proximal the first outlet passage of the housing and a distal end located distal to the first distal passage of the housing.

9. The suture retention device according to claim 1, wherein when the first actuator is in the first undepressed position and the second actuator is in the second undepressed position, a top of the second actuator is located above a top of the first actuator.

10. The suture retention device according to claim 1, wherein the first actuator is configured to rotate inside the first cavity between a first angular position when in the first fully depressed position and a second angular position different from the first angular position when in the first undepressed position.

11. The suture retention device according to claim 1, wherein the second actuator is coupled to the first actuator and rotates with the first actuator.

12. The suture retention device according to claim 1, wherein the second actuator is configured to rotate inside the second cavity between a first angular position when in the second fully depressed position and a second angular position different from the first angular position when in the second undepressed position.

13. The suture retention device according to claim 10, wherein the housing and the first actuator are configured such that when the first actuator is in the second angular position, the one or more end portions of the suture, when situated in the suture retention device, is/are gripped between the housing and the first actuator.

14. The suture retention device according to claim 12, wherein the first and second actuators are configured such that when the second actuator is in the second angular position, the one or more end portions of the suture, when situated in the suture retention device, is/are gripped between the first and second actuators.

15. The suture retention device according to claim 10, wherein the first actuator includes one or more protrusions protruding from a side surface, and an internal wall of the housing includes a respective one or more angular grooves in which the one or more protrusions reside, the one or more protrusions and the one or more angular grooves being configured to guide the first actuator between the first and second angular positions.

16. The suture retention device according to claim 10, wherein an internal wall of the housing includes one or more protrusions, the first actuator includes a side surface having one or more angular grooves in which the one or more protrusions reside, the one or more protrusions and the one or more angular grooves being configured to guide the first actuator between the first and second angular positions.

17. The suture retention device according to claim 15, wherein the one or more protrusions are snap-fitted into the respective one or more grooves.

18. The suture retention device according to claim 16, wherein the one or more protrusions are snap-fitted into the respective one or more grooves.

19. The suture retention device according to claim 3, wherein a portion of the housing and/or a portion of the first actuator comprises a roughened surface in a region of one or both of the first and second locations.

20. The suture retention device according to claim 4, wherein a portion of the first actuator and/or a portion of the second actuator comprises a roughened surface in a region of one or both of the third and fourth locations.
